(19) United States
(12) Patent Application Publication
(10) Pub. No.: US 2017/0231223 A1
(43) Pub. Date: Aug. 17, 2017

(54) Organic Ammonium Salts Of Anionic Pesticides

(71) Applicant: Clariant International Ltd., Muttenz (CH)

(72) Inventors: Peter KLUG, Grossostheim (DE); Peter BAUR, Schondorf (DE); Martin BAUER, Frankfurt am Main (DE)

(73) Assignee: Clariant International Ltd., Muttenz (CH)

(21) Appl. No.: 15/502,993

(22) PCT Filed: Aug. 12, 2015
(86) PCT No.: PCT/EP2015/068602

§ 371 Date: Feb. 9, 2017

(30) Foreign Application Priority Data
Aug. 13, 2014 (DE) 102014012022.8

(51) Int. Cl. A01N 37/40; A01N 25/02; A01N 39/04; A01N 37/02; C07C 59/64; C07D 401/12; C07C 215/06; C07C 213/08; C07C 59/70; C07C 53/126; C07C 317/24; C07C 315/04; A01N 47/38; C07C 51/41

(52) U.S. Cl. CPC A01N 37/40; A01N 47/38; A01N 25/02; A01N 39/04; A01N 37/02; C07C 59/64; C07C 51/412; C07C 215/06; C07C 213/08; C07C 59/70; C07C 53/126; C07C 317/24; C07C 315/04; C07D 401/12; C07C 2601/14

(57) ABSTRACT

The invention relates to an organic ammonium salt of formula (I), wherein the symbols have the following meanings: A- is an anionic pesticide, R¹ is H, C₁C₄-alkyl, CH₂CH₂OH or CH₂CH(CH₃)OH; R is H, CH₃ or two adjacent radicals R together form a group —C(R′)— and R′ is equal to or different from H or CH₃, wherein said salt is suitable for reducing the volatility of active substances in the spray application of anionic pesticides.

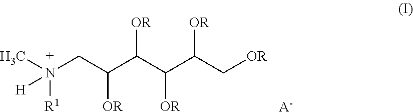

The invention relates to organic ammonium salts of anionic pesticides, to a process for preparation thereof, to agrochemical compositions comprising these salts, and to methods of controlling harmful organisms using the salts and compositions mentioned.

Pesticides (particularly herbicides, fungicides and insecticides) are chemical substances, prepared synthetically or of natural origin, which penetrate into plant cells or tissue or into parasitic organisms in or on the plant and damage and/or destroy them. The majority of pesticides are herbicides. Pesticides are typically used in the form of liquid or solid concentrated preparations (formulations) in agriculture. These are easier for the user to handle or ensure higher efficacy of the active ingredient. The formulations are typically diluted with water prior to use and then deployed by spray application.

Water-soluble concentrates (soluble liquids, abbreviated to SL) are an important form of pesticide preparations. They play a major role particularly in the case of herbicides, the pesticides often being used in the form of water-soluble salts which are converted to their alkali metal or ammonium salts by neutralization of the acid form of the herbicides with suitable bases. Under some circumstances, a second water-insoluble active ingredient is present in the pesticide preparation. In that case, the preparation is a suspension concentrate (SC), even when an active ingredient is dissolved in the aqueous phase.

A particularly important role is played by the water-soluble salts of herbicides, for example of glyphosate or glufosinate or of the auxin herbicides such as clodinafop, 2,4-D, MCAP or quinclorac. They are preferably used as the alkali metal salt or in the form of various ammonium salts or as a mixture of these salts, usually as aqueous formulations.

In the use of pesticides, it is advantageous when they have a low volatility, since a high volatility is especially associated with increased drift of relatively small spray droplets of droplet diameter below 150 mm of the pesticides, with high losses and ingress into the non-target vegetation on spraying. Such drift is undesirable for environmental and economic reasons, since unintended damage could be caused and the effect of the pesticides on the target organisms is lowered.

In order to avoid this effect, the use of volatile pesticides that are in the form of free acids in salt form is known.

U.S. Pat. No. 4,405,531 and WO 97/24931 disclose various organic salts of glyphosate with di- and polyamines.

U.S. Pat. No. 5,221,791 describes aminoalkylpyrrolidone salts of pesticides such as dicamba.

EP-A 0 375 624 discloses nonvolatile salts of pesticides with various polyamines.

EP-A 0 183 384 describes nonvolatile salts of dicamba with amino alcohols.

Even though good results are already achieved with the known systems, there is still a great deal of room for improvement, especially with regard to an increase in the water solubility of the active ingredients in the concentrate, lower volatility of the active ingredients and improved efficacy through better uptake.

It has now been found that the salts of mono- and dialkylglucamines with anionic pesticides are particularly suitable for use as aqueous spray applications.

The invention therefore provides an organic ammonium salt of the formula (I)

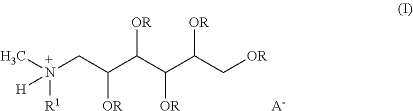

where the symbols have the following definitions:
(A)⁻ is an anionic pesticide,
R¹ is H, C₁-C₄-alkyl, CH₂CH₂OH or CH₂CH(CH₃)OH;
R is H, CH₃, or two adjacent R radicals together form a —C(R′)— group and
R′ is the same or different and is H or CH₃.

The invention further provides a process for preparing an ammonium salt of the invention, wherein the protonated form of an anionic herbicide is reacted with a glucamine of the formula (II)

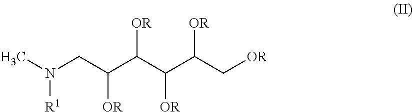

in which the symbols have the definitions given in the formula (I).

The invention further provides for the use of the ammonium salts of the invention as pesticides.

The invention likewise provides a pesticide composition comprising
a) one or more ammonium salts (I) of the invention and
b) one or more formulation auxiliaries.

The invention likewise provides a method of controlling harmful organisms, wherein the harmful organism or its habitat is brought into contact with an ammonium salt (I) of the invention or a pesticide composition of the invention.

The invention further provides for the use of an ammonium salt of the invention for reducing the volatility of the anionic pesticide present in salt form.

The ammonium salts (I) of the invention are of excellent suitability for use as aqueous spray applications and especially exhibit good water solubility and reduced volatility without elevated drift on use.

The cation of the ammonium salts (I) of the invention results from protonation of a glucamine (II)

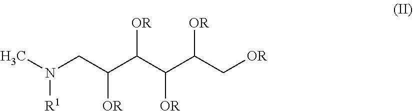

where the symbols have the following definitions:
R¹ is H, C₁-C₄-alkyl, CH₂CH₂OH or CH₂CH(CH₃)OH;
R is H, CH₃, or two adjacent R radicals together form a —C(R′)— group and
R′ is the same or different and is H or CH₃.

Preferably, the symbols in the formulae (I) and (II) have the following definitions:
R¹ is preferably H, C₁-C₄-alkyl or CH₂CH₂OH.
R is preferably H or CH₃.

Preference is given to cations in which all the symbols have the preferred definitions.

More preferably, the symbols in the formulae (I) and (II) have the following definitions:
R¹ is more preferably H, CH₃ or CH₂CH₂OH.
R is more preferably H.

Particular preference is given to cations in which all the symbols have the particularly preferred definitions.

Very particular preference is given to protonated dimethylglucamine (R¹=CH₃, R=H) and hydroxyethyl-glucamine (R¹=CH₂CH₂OH, R=H).

The pentahydroxyhexyl radical in the alkylglucamines of the formula (I) and (II) has various chiral centers, such that several stereoisomers can exist in each case. Typically, the alkylglucamines of the formula (II) are prepared from naturally occurring sugars, such as D-glucose, but the use of other natural or synthetic hexoses or other C₆ units is also possible in principle, such that different stereoisomers of the formulae (I) and (II) can be the result.

The preparation of the alkylglucamines of the formula (II) has been sufficiently described before and is known to the person skilled in the art. It is effected (for compounds with R¹=C₁-C₄-alkyl), for example, as described in EP-A 1 676 831 by reductive alkylation of N-alkylpolyhydroxylamines with aldehydes or ketones in the presence of hydrogen and a transition metal catalyst.

Dimethylglucamine can be prepared, for example, according to EP-A 0 614 881. Dimethylglucamine is usable in the form of a 50% aqueous solution. Hydroxyethyl-N-methylglucamine can be prepared by reaction of N-methylglucamine with ethylene oxide in aqueous solution. Dimethylglucamine and hydroxyethyl-N-methylglucamine, being tertiary amines, are less prone to the formation of nitrosamines and are correspondingly preferred.

Compounds in which two adjacent R radicals together form a —C(R′)— group can be obtained by formation of the corresponding acetonide or acetal with acetone or with acetaldehyde or formaldehyde.

The alkylglucamines of the formula (II) are preferably based on renewable raw materials and feature an advantageous toxicological and environmental profile.

The (A)⁻ group in the formula (I) is an anionic pesticide.

The anionic pesticide is preferably the conjugate base of a pesticidal Bronsted acid having a pKₐ of 1.5 to 7, preferably 2 to 6 and more preferably 2.5 to 5.5.

Preferably, the conjugate base of the anionic pesticide has at least one carboxylic acid, thiocarboxylic acid, sulfonic acid, thiosulfonic acid, sulfinic acid, phosphonic acid, sulfonylurea, sulfonicarbamate, phenol, hydrobenzonitrile, keto-enol and/or triketone group. Preference is given to carboxylic acid and phosphonic acid groups.

Preferred pesticides include anionic herbicides, plant growth regulators, insecticides, acaricides, fungicides, bactericides, nematicides, plant nutrients and repellents. Preference is given to anionic herbicides, growth regulators and insecticides, especially herbicides.

Preference is given to the following anionic pesticides from the group of the herbicides: acifluorfen, aminocyclopyrachlor, aminopyralid, amitrol, asulam, benazolin, bensulfuron, bentazon, bialafos, bispyribac, bromacil, bromoxynil, bicyclopyron, chlorflurenol, chlorthal, clodinafop, cloprop, clopyralid, 4-CPA, 4-CPB, 4-CPP, cyhalofop, 2,4-D, dalapon, 2,4-DB, 3,4-DA, 3,4-DB, 3,4-DP, 2,4,5-T, 2,4,5-TB, dicamba, dichlorprop, dichlorprop-P, diflufenzopyr, diclofop, endothal, fenoprop, fenoxaprop, fenthiaprop, flamprop, flamprop-M, florasulam, fluazifop, fluazifop-P, flucarbazone, flufenpyr, flumiclorac, fluoroglycofen, flupropanate, flurenol, fluroxypyr, fluthiacet, fomesafen, foramsulfuron, fosamine, glufosinate, glufosinate-P, glyphosate, halosulfuron, haloxyfop, haloxyfop-P, imizameth, imazamethabenz, imazamox, imazapic, imazapyr, imazaquin, imazethapyr, isoxapyrifop, lactofen, MCPA, MCPA-thioethyl, MCPB, mecoprop, mecoprop-P, mesosulfuron, mesotrione, nicosulfuron, octanoic acid, oleic acid, pelargonic acid, picloram, primisulfuron, propaquizafop, propoxycarbazone, pyraflufen, pyrithiobac, pyrithiobac, pyroxsulam, quinclorac, quinmerac, quizalofop, quizalofop-P, sulcotrione, sulfometuron, 2,3,6-TBA, TCA, tembotrione, thiencarbazone, topramezone, tricamba, triclopyr and trifopsic acid.

Preference is further given to the following anionic pesticides from the group of the growth regulators: abscisic acid, glyphosine, indole-3-acetic acid, indole-4-butyric acid, jasmonic acid, 1-naphthylacetic acid, 2-naphthylacetic acid, naphthyloxyacetic acid, prohexadione, salicylic acid, 2,3,5-triiodobenzoic acid and trinexapac.

Preference is also given to the following anionic pesticides from the group of the insecticides: thuringiensin, insecticidal free keto-enols such as spirotetramat (free enol) and pinoxaden. Preference is likewise given to the following anionic pesticides from the group of the fungicides: fosetyl, polyoxorim and polyoxin D.

Particularly preferred anionic pesticides are bicyclopyron, bromoxynil, clopyralid, 2,4-D (2,4-dichlorophenoxyacetic acid), dicamba, diquat, formasafen, florasulam, fomesafen, glufosinate, glyphosate, MCPA, mesotrione, nicosulfuron, pelargonic acid, quinclorac, quinmerac, salicylic acid, sulcotrione, tembotrione and triclopyr.

Very particular preference is given to bromoxynil, clopyralid, 2,4-D, dicamba, fomesafen, MCPA, pelargonic acid, sulcotrione and triclopyr.

Especially preferred are bromoxynil, clopyralid, 2,4-D, dicamba and pelargonic acid.

Preferred, particularly preferred, very particularly preferred and especially preferred are also the dimethylglucammonium salts of the preferred, particularly preferred, very particularly preferred and especially preferred anionic pesticides A⁻.

Said pesticides are known and commercially available.

They are described, for example, in Weed Research 26 (1986) 441-445 or “The Pesticide Manual”, 16th edition, The British Crop Protection Council and the Royal Society of Chemistry, 2012 and the literature cited therein.

The ammonium salts (I) of the invention exhibit essentially the same spectrum of action as the corresponding pesticides A and can thus be used to counter the same target organisms known to the person skilled in the art.

With the ammonium salts (I) of the invention, it is possible to produce pesticide compositions of the invention, especially aqueous herbicide compositions, with excellent action and excellent performance properties, such as reduced volatility without altered drift.

The pesticide compositions comprise
a) one or more ammonium salts (I) of the invention, as described above, and
b) one or more formulation auxiliaries.

In a preferred embodiment of the invention, the amount of the one or more ammonium salts (I) a) in the compositions of the invention is more than 100 g/L, preferably more than 200 g/L and more preferably more than 300 g/L. These figures are based on the total weight of the pesticide composition of the invention and on the amount of free acid (i.e. of the protonated form), called the acid equivalent (a.e.), of the anionic pesticides A.

The formulation auxiliaries (b) are, for example, solvents, surfactants, defoamers, functional polymers, adjuvants, antifreezes, preservatives, dyes and/or ammonium salts.

A preferred solvent is water. Preference is also given to the use of one or more cosolvents. The cosolvents may be a single solvent or a mixture of two or more solvents. Suitable solvents for this purpose are all polar solvents which are compatible with the aqueous pesticide composition and form a homogeneous phase. Suitable cosolvents are, for example, monohydric alcohols such as methanol, ethanol, propanols, butanols, benzyl alcohol or further polyhydric alcohols such as ethylene glycol, diethylene glycol, propylene glycol, dipropylene glycol, glycerol or polyglycols such as polyethylene glycols, polypropylene glycols or mixed polyalkylene glycols (PAGs). Further suitable solvents are ethers, for example propylene glycol mono- or dimethyl ether, dipropylene glycol mono- or dimethyl ether, amides, for example N-methyl- or N-ethylpyrrolidone, or N,N-dimethyllactamide, -caprylamide or -decanamide.

The surfactants may generally be any nonionic, amphoteric, cationic or anionic surfactants that are compatible with the composition.

Examples of nonionic surfactants are glucamides, especially as described in WO 2014/067663, ethoxylates and alkoxylates of longer-chain aliphatic or aromatic alcohols, fatty amine ethoxylates, longer-chain ether amine alkoxylates, (optionally ethoxylated) sorbitan esters, alkyl polyglycosides. Suitable amphoteric surfactants include long-chain alkyl dimethyl betaines or alkyl dimethylamine oxides, or alkyl dimethylamine amidopropylamine oxides. Among the anionic surfactants, suitable examples are ether sulfates of ethoxylated fatty alcohols, reaction products of (optionally ethoxylated) long-chain alcohols with phosphoric acid derivatives. “Long-chain” is understood to mean linear or branched hydrocarbon chains having at least 6 and at most 22 carbon atoms.

Suitable defoamers are fatty acid alkyl ester alkoxylates, organopolysiloxanes such as polydimethylsiloxanes and mixtures thereof with microfine, optionally silanized silica; perfluoroalkyl-phosphonates and -phosphinates, paraffins, waxes and microcrystalline waxes, and mixtures thereof with silanized silica. Also advantageous are mixtures of various foam inhibitors, for example those formed from silicone oil, paraffin oil and/or waxes. Preference is given to the defoamers described in German patent application DE 10 2014 208 244.7.

The functional polymers which may be present in the pesticide composition of the invention are high molecular weight compounds of synthetic or natural origin having a molar mass of greater than 10 000. The functional polymers may, for example, act as antidrift agents or enhance rain resistance.

In a further embodiment of the invention, the pesticide compositions of the invention comprise one or more adjuvants as usable in a known manner in aqueous pesticide compositions.

These are preferably fatty amine ethoxylates, ether amine ethoxylates, alkyl betaines or amidoalkyl betaines, amine oxides or amidoalkylamine oxides, alkyl polyglycosides or copolymers of glycerol, coconut fatty acid and phthalic acid.

These adjuvants are known from the literature and are described, for example, in WO 2009/029561.

Further preferred adjuvants are glucamides, especially as described in WO 2014/067663.

Likewise preferred as adjuvants are drift-reducing polyglycerol esters as described in WO 2014/063818, which comprise one or more copolymers A), where the copolymers contain one or more structural units derived from
a) 19.9% to 75.9% by weight of glycerol
b) 0.1% to 30% by weight of at least one dicarboxylic acid and
c) 24% to 80% by weight of at least one monocarboxylic acid of formula (III)

R²—COOH (III)
where R² is (C₅-C₂₉)-alkyl; (C₇-C₂₉)-alkenyl; phenyl or naphthyl.

Preservatives used may be organic acids and their esters, for example ascorbic acid, ascorbyl palmitate, sorbate, benzoic acid, methyl and propyl 4-hydroxybenzoate, propionates, phenol, for example 2-phenylphenate, 1,2-benzisothiazolin-3-one, formaldehyde, sulfurous acid and salts thereof.

Suitable antifreezes are, for example, ethylene glycol, propylene glycol, glycerol and urea.

Suitable ammonium salts are water-soluble ammonium salts such as ammonium sulfate, ammonium nitrate, ammonium nitrate urea, ammonium phosphate, ammonium citrate, ammonium thiosulfate and/or ammonium chloride, preferably ammonium sulfate, ammonium nitrate, ammonium citrate and/or ammonium nitrate urea, more preferably ammonium sulfate.

As well as the ammonium salts (I) a) and formulation auxiliaries b), the pesticide compositions may comprise further pesticides c).

Examples of pesticides c) which can constitute combination partners for the ammonium salts (I) are given hereinafter. Where the pesticides are anionic pesticides, these can of course also be used in the form of ammonium salts of the formula (I).

Examples of herbicides include:

Active ingredients based on inhibition of, for example, acetolactate synthase, acetyl-CoA carboxylase, cellulose synthase, enolpyruvylshikimate-3-phosphate synthase, glutamine synthetase, p-hydroxyphenylpyruvate dioxygenase, phytoendesaturase, photosystem photosystem II, protoporphyrinogen oxidase usable, as described, for example, from Weed Research 26 (1986) 441 445 or “The Pesticide Manual”, 16th edition, The British Crop Protection Council and the Royal Soc. of Chemistry, 2012 and literature cited therein. Known herbicides or plant growth regulators which can be combined with the compounds of the invention include, for example, the following active ingredients (the compounds are referred to either by the common name according to the International Organization for Standardization (ISO) or by the chemical name or by the code number) and always encompass all use forms such as acids, salts, esters and isomers such as stereoisomers and optical isomers. This list includes, by way of example, one use form and in some cases also a plurality of use forms:
acetochlor, acibenzolar, acibenzolar-S-methyl, aclonifen, alachlor, allidochlor, alloxydim, alloxydim-sodium, ametryn, amicarbazone, amidochlor, amidosulfuron, aminocyclopyrachlor-potassium, aminocyclopyrachlor-methyl, ammonium sulfamate, ancymidol, anilofos, atrazine, aviglycin, azafenidin, azimsulfuron, aziprotryn, beflubutamid, benazolinethyl, bencarbazone, benfluralin, benfuresate, bensulide, benzfendizone, benzobicyclon, benzofenap, benzofluor, benzoylprop, benzyladenin, bromobutide, bromofenoxim, bromuron, buminafos, busoxinone, butachlor, butafenacil, butamifos, butenachlor, butralin, butroxydim, butylate, cafenstrole, carbaryl, carbetamide, carvone, chlorocholine chloride, chlomethoxyfen, chlorbromuron, chlorbufam, chlorfenac, chlorfenac-sodium, chlorfenprop, chlorflurenol-methyl, chloridazon, chlorimuron-ethyl, chlormequat chloride, chlornitrofen, 4-chlorophenoxyacetic acid, chlorophthalim, chlorpropham, chlorotoluron, chlorsulfuron, cinidon, cinidonethyl, cinmethylin, cinosulfuron, clethodim, clodinafop-propargyl, clofencet, clomazone, clomeprop, cloprop, cloransulam, cloransulam-methyl, cloxyfonac, cumyluron, cyanamide, cyanazine, cyclanilide, cycloate, cyclosulfamuron, cycloxydim, cycluron, cyperquat, cyprazine, cyprazole, cytokinine, daimuron/dymron, daminozide, dazomet, n-decanol, desmedipham, desmetryn, detosyl pyrazolate (DTP), diallate, diaminozid, dichlobenil, diclosulam, diethatyl, diethatyl-ethyl, difenoxuron, diflufenican, diflufenzopyr-sodium, dikegulac-sodium, dimefuron, dimepiperate, dimethachlor, dimethametryn, dimethenamid, dimethenamid-P, dimethipin, dimetrasulfuron, dinitramine, dinoseb, dinoterb, diphenamid, diisopropylnaphthalene, dipropetryn, dithiopyr, diuron, DNOC, eglinazine-ethyl, EPTC, esprocarb, ethalfluralin, ethametsulfuron, ethametsulfuron-methyl, ethylnaphthyl acetate, ethephon, ethidimuron, ethiozin, ethofumesate, ethoxyfen, ethoxyfen-ethyl, ethoxysulfuron, etobenzanid, F-5331, i.e. N-[2-chloro-4-fluoro-5-[4-(3-fluoropropyl)-4,5-dihydro-5-oxo-1H-tetrazol-1-yl]-phenyl]ethanesulfonamide, F-7967, i.e. 3-[7-chloro-5-fluoro-2-(trifluoromethyl)-1H-benzimidazol-4-yl]-1-methyl-6-(trifluoromethyl)pyrimidine-2,4(1H,3H)-dione, fenoxasulfone, fentrazamide, fenuron, flazasulfuron, fluazolate, flucarbazone-sodium, flucetosulfuron, fluchloralin, flufenacet (thiafluamide), flumetralin, flumetsulam, flumioxazin, flumipropyn, fluometuron, fluorodifen, flupoxam, flupropacil, flupyrsulfuron, flupyrsulfuron-methyl-sodium, fluridone, flurochloridone, flurprimidol, flurtamone, fluthiacet-methyl, fluthiamide, forchlorfenuron, furyloxyfen, gibberellic acid, H-9201, i.e. O-(2,4-dimethyl-6-nitrophenyl)-O-ethylisopropylphosphorus amidothioate, halosafen, halosulfuron-methyl, hexazinone, HW-02, i.e. 1-(dimethoxyphosphoryl)ethyl (2,4-dichlorophenoxy)-acetate, imazosulfuron, inabenfide, indanofan, indaziflam, iodosulfuron, iodosulfuronmethyl-sodium, iofensulfuron, iofensulfuron-sodium, ioxynil, ipfencarbazone, isocarbamid, isopropalin, isoproturon, isouron, isoxaben, isoxachlortole, isoxaflutole, KUH-043, i.e. 3-({[5-(difluoromethyl)-1-methyl-3-(trifluoromethyl)-1H-pyrazol-4-yl]methyl}sulfonyl)-5,5-dimethyl-4,5-dihydro-1,2-oxazole, karbutilate, ketospiradox, lenacil, linuron, maleic hydrazide, mefenacet, mefluidide, mepiquat chloride, methabenzthiazuron, metam, metamitron, metazachlor, metazasulfuron, methazole, methiopyrsulfuron, methiozolin, methoxyphenone, methyldymron, 1-methylcyclopropene, methyl isothiocyanate, metobenzuron, metobromuron, metolachlor, S-metolachlor, metosulam, metoxuron, metribuzin, metsulfuron, metsulfuron-methyl, molinate, monalide, monocarbamide, monocarbamide dihydrogensulfate, monolinuron, monosulfuron, monosulfuron ester, monuron, MT-128, i.e. 6-chloro-N-[(2E)-3-chloroprop-2-en-1-yl]-5-methyl-N-phenylpyridazin-3-amine, MT-5950, i.e. N-[3-chloro-4-(1-methylethyl)phenyl]-2-methylpentanamide, NGGC-011, naproanilide, napropamide, NC-310, i.e. 4-(2,4-dichlorobenzoyl)-1-methyl-5-benzyloxypyrazole, neburon, nipyraclofen, nitralin, nitrofen, nitroguaiacolate, nitrophenolate-sodium (isomer mixture), nitrofluorfen, nonanoic acid, norflurazon, orbencarb, orthosulfamuron, oryzalin, oxadiargyl, oxadiazon, oxasulfuron, oxaziclomefone, oxyfluorfen, paclobutrazol, paraquat, paraquat dichloride, pendimethalin, pendralin, penoxsulam, pentanochlor, pentoxazone, perfluidone, pethoxamid, phenisopham, phenmedipham, phenmedipham-ethyl, picolinafen, pinoxaden, piperophos, pirifenop, pirifenop-butyl, pretilachlor, probenazole, profluazol, procyazine, prodiamine, prifluraline, profoxydim, prohexadione-calcium, prohydrojasmone, prometon, prometryn, propachlor, propanil, propazine, propham, propisochlor, propyrisulfuron, propyzamide, prosulfalin, prosulfocarb, prosulfuron, prynachlor, pyraclonil, pyrasulfotole, pyrazolynate (pyrazolate), pyrazosulfuron, pyrazosulfuron-ethyl, pyrazoxyfen, pyribambenz, pyribambenz-isopropyl, pyribambenz-propyl, pyribenzoxim, pyributicarb, pyridafol, pyridate, pyriftalid, pyrimisulfan, pyroxasulfone, pyroxsulam, quinoclamine, rimsulfuron, saflufenacil, secbumeton, sethoxydim, siduron, simazine, simetryn, SN-106279, i.e. methyl (2R)-2-({7-[2-chloro-4-(trifluoromethyl)-phenoxy]-2-naphthyl}oxy)propanoate, sulfallate (CDEC), sulfentrazone, sulfo-sulfuron, SW-065, SYN-523, SYP-249, i.e. 1-ethoxy-3-methyl-1-oxobut-3-en-2-yl 5-[2-chloro-4-(trifluoromethyl)phenoxy]-2-nitrobenzoate, SYP-300, i.e. 1-[7-fluoro-3-oxo-4-(prop-2-yn-1-yl)-3,4-dihydro-2H-1,4-benzoxazin-6-yl]-3-propyl-2-thioxo-imidazolidine-4,5-dione, tebutam, tebuthiuron, tecnazene, tefuryltrione, tepraloxydim, terbacil, terbucarb, terbuchlor, terbumeton, terbuthylazine, terbutryn, thenylchlor, thiafluamide, thiazafluron, thiazopyr, thidiazimin, thidiazuron, thiencarbazone-methyl, thifensulfuron, thifensulfuron-methyl, thiobencarb, tiocarbazil, tralkoxydim, triafamone, triallate, triasulfuron, triaziflam, triazofenamide, tribenuron, tribenuron-methyl, tribufos, tridiphane, trietazine, trifloxysulfuron, trifloxysulfuron-sodium, trifluralin, triflusulfuron, triflusulfuron-methyl, trimeturon, trinexapac-ethyl, tritosulfuron, tsitodef, uniconazole, uniconazole-P, vernolate, ZJ-0862, i.e. 3,4-dichloro-N-{2-[(4,6-dimethoxypyrimidin-2-yl)oxy]-benzyl}aniline.

Examples of plant growth regulators further include natural plant hormones such as esters of salicylic acid, kinetin and brassinosteroids.

Further substances that should be mentioned are those which can act as plant growth regulators and/or plant fortifiers, in order to reduce the effect of stress factors such as heat, cold, drought, salt, oxygen deficiency or flooding on plant growth. Examples of these include glycine betaine (betaine), choline, potassium phosphate or other phosphate salts, and silicates.

Examples of plant nutrients include customary inorganic or organic fertilizers for supplying plants with macro- and/or micronutrients.

Examples of fungicides include:
(1) Ergosterol biosynthesis inhibitors, for example aldimorph, azaconazole, bitertanol, bromuconazole, cyproconazole, diclobutrazole, difenoconazole, diniconazole, diniconazole-M, dodemorph, dodemorph acetate, epoxiconazole, etaconazole, fenarimol, fenbuconazole, fenhexamid, fenpropidin, fenpropimorph, fluquinconazole, flurprimidol, flusilazole, flutriafol, furconazole, furconazole-cis, hexaconazole, imazalil, imazalil sulfate, imibenconazole, ipconazole, metconazole, myclobutanil, naftifin, nuarimol, oxpoconazole, paclobutrazole, pefurazoate, penconazole, piperalin, prochloraz, propiconazole, prothioconazole, pyributicarb, pyrifenox, quinconazole, simeconazole, spiroxamine, tebuconazole, terbinafine, tetraconazole, triadimefon, triadimenol, tridemorph, triflumizole, triforine, triticonazole, uniconazole, uniconazole-p, viniconazole, voriconazole, 1-(4-chlorophenyl)-2-(1H-1,2,4-triazol-1-yl)cycloheptanol, methyl 1-(2,2-dimethyl-2,3-dihydro-1H-inden-1-yl)-1H-imidazole-5-carboxylate, N′-{5-(difluoromethyl)-2-methyl-4-[3-(trimethylsilyl)propoxy]phenyl}-N-ethyl-N-methylimido-formamide, N-ethyl-N-methyl-N′-{2-methyl-5-(trifluoromethyl)-4-[3-(trimethylsilyl)propoxy]phenyl}-imidoformamide and O-[1-(4-methoxyphenoxy)-3,3-dimethylbutan-2-yl] 1H-imidazole-1-carbothioate.
(2) Respiration inhibitors (respiratory chain inhibitors), for example bixafen, boscalid, carboxin, diflumetorim, fenfuram, fluopyram, flutolanil, fluxapyroxad, furametpyr, furmecyclox, isopyrazam mixture of the syn-epimeric racemate 1RS,4SR,9RS and of the anti-epimeric racemate 1RS,4SR,9SR, isopyrazam (anti-epimeric racemate), isopyrazam (anti-epimeric enantiomer 1R,4S,9S), isopyrazam (anti-epimeric enantiomer 1S,4R,9R), isopyrazam (syn-epimeric racemate 1RS,4SR,9RS), isopyrazam (syn-epimeric enantiomer 1R,4S,9R), isopyrazam (syn-epimeric enantiomer 1S,4R,9S), mepronil, oxycarboxin, penflufen, penthiopyrad, sedaxane, thifluzamid, 1-methyl-N-[2-(1,1,2,2-tetrafluoroethoxy)phenyl]-3-(trifluoromethyl)-1H-pyrazole-4-carboxamide, 3-(difluoromethyl)-1-methyl-N-[2-(1,1,2,2-tetrafluoroethoxy)phenyl]-1H-pyrazole-4-carboxamide, 3-(difluoromethyl)-N-[4-fluoro-2-(1,1,2,3,3,3-hexafluoropropoxy)phenyl]-1-methyl-1H-pyrazole-4-carboxamide, N-[1-(2,4-dichlorophenyl)-1-methoxypropan-2-yl]-3-(difluoromethyl)-1-methyl-1H-pyrazole-4-carboxamide, 5,8-difluoro-N-[2-(2-fluoro-4-1[4-(trifluoromethyl)pyridin-2-yl]oxylphenyl)ethyl]-quinazoline-4-amine, N-[9-(dichloromethylene)-1,2,3,4-tetrahydro-1,4-methanonaphthalen-5-yl]-3-(difluoro-methyl)-1-methyl-1H-pyrazole-4-carboxamide, [(1S,4R)-9-(dichloromethylene)-1,2,3,4-tetrahydro-1,4-methanonaphthalen-5-yl]-3-(difluoromethyl)-1-methyl-1H-pyrazole-4-carboxamide and N-[(1R,4S)-9-(dichloro-methylene)-1,2,3,4-tetrahydro-1,4-methanonaphthalen-5-yl]-3-(difluoromethyl)-1-methyl-1H-pyrazole-4-carboxamide.
(3) Respiration inhibitors (respiratory chain inhibitors) acting on complex III of the respiratory chain, for example ametoctradin, amisulbrom, azoxystrobin, cyazofamid, coumethoxystrobin, coumoxystrobin, dimoxystrobin, enestroburin, famoxadone, fenamidone, fenoxystrobin, fluoxastrobin, kresoxim-methyl, metominostrobin, orysastrobin, picoxystrobin, pyraclostrobin, pyrametostrobin, pyraoxystrobin, pyribencarb, triclopyricarb, trifloxystrobin, (2E)-2-(2-{[6-(3-chloro-2-methyl-phenoxy)-5-fluoropyrimidin-4-yl]oxylphenyl)-2-(methoxy-imino)-N-methylethanamide, (2E)-2-(methoxyimino)-N-methyl-2-(2-1[({(1E)-1-[3-(trifluoromethyl)phenyl]-ethylidenelamino)oxy]methyllphenyl)ethanamide, (2E)-2-(methoxyimino)-N-methyl-2-{2-[(E)-({1-[3-(trifluoromethyl)phenyl]ethoxy}imino)methyl]phenyl}-ethanamide, (2E)-2-(2-[({[(1E)-1-(3-([(E)-1-fluoro-2-phenylethenyl]oxy}phenyl)ethylidene]amino}oxy)methyl]-phenyl}-2-(methoxyimino)-N-methylethanamide, (2E)-2-{2-[({[(2E,3E)-4-(2,6-dichlorophenyl)but-3-en-2-ylidene]-amino}oxy)methyl]phenyl}-2-(methoxyimino)-N-methyl-ethanamide, 2-chloro-N-(1,1,3-trimethyl-2,3-dihydro-1H-inden-4-yl)pyridine-3-carboxamide, 5-methoxy-2-methyl-4-(2-{[({(1E)-1-[3-(trifluoromethyl)phenyl]ethylidenel-amino)oxy]methyl}phenyl)-2,4-dihydro-3H-1,2,4-triazol-3-one, methyl (2E)-2-[({cyclopropyl[(4-methoxy-phenyl)imino]methyl}sulfanyl)methyl]phenyl}-3-methoxy-prop-2-enoate, N-(3-ethyl-3,5,5-trimethylcyclohexyl)-3-(formylamino)-2-hydroxybenzamide, 2-{2-[(2,5-dimethyl-phenoxy)methyl]phenyl}-2-methoxy-N-methylacetamide and (2R)-2-{2-[(2,5-dimethylphenoxy)methyl]phenyl}-2-methoxy-N-methylacetamide.
(4) Mitosis and cell division inhibitors, for example benomyl, carbendazim, chlorfenazole, diethofencarb, ethaboxam, fluopicolide, fuberidazole, pencycuron, thiabendazole, thiophanate-methyl, thiophanate, zoxamide, 5-chloro-7-(4-methylpiperidin-1-yl)-6-(2,4,6-trifluorophenyl)[1,2,4]triazolo[1,5-a]pyrimidine and 3-chloro-5-(6-chloropyridin-3-yl)-6-methyl-4-(2,4,6-trifluorophenyl)pyridazine.
(5) Compounds with multisite activity, for example Bordeaux mixture, captafol, captan, chlorothalonil, copper preparations such as copper hydroxide, copper naphthenate, copper oxide, copper oxychloride, copper sulfate, dichlofluanid, dithianon, dodine, dodine free base, ferbam, fluorofolpet, folpet, guazatine, guazatine acetate, iminoctadine, iminoctadine albesilate, iminoctadine triacetate, mancopper, mancozeb, maneb, metiram, metiram zinc, oxine-copper, propamidine, propineb, sulfur and sulfur preparations, for example calcium polysulfide, thiram, tolylfluanid, zineb and ziram.
(6) Resistance inductors, for example acibenzolar-S-methyl, isotianil, probenazole and tiadinil.
(7) Amino acid and protein biosynthesis inhibitors, for example andoprim, blasticidin-S, cyprodinil, kasugamycin, kasugamycin hydrochloride hydrate, mepanipyrim, pyrimethanil and 3-(5-fluoro-3,3,4,4-tetramethyl-3,4-dihydroisoquinolin-1-yl)quinoline.
(8) Inhibitors of ATP production, for example fentin acetate, fentin chloride, fentin hydroxide and silthiofam.
(9) Cell wall synthesis inhibitors, for example benthiavalicarb, dimethomorph, flumorph, iprovalicarb, mandipropamid, validamycin A and valifenalate.
(10) Lipid and membrane synthesis inhibitors, for example biphenyl, chloroneb, dicloran, edifenphos, etridiazole, iodocarb, iprobenfos, isoprothiolane, propamocarb, propamocarb hydrochloride, prothiocarb, pyrazophos, quintozene, tecnazene and tolclofos-methyl.
(11) Melanin biosynthesis inhibitors, for example carpropamid, diclocymet, fenoxanil, fthalide, pyroquilon, tricyclazole and 2,2,2-trifluoroethyl {3-methyl-1-[(4-methylbenzoyl)amino]butan-2-yl}carbamate.
(12) Nucleic acid synthesis inhibitors, for example benalaxyl, benalaxyl-M (kiralaxyl), bupirimate, clozylacon, dimethirimol, ethirimol, furalaxyl, hymexazol, metalaxyl, metalaxyl-M (mefenoxam), ofurace, oxadixyl and oxolinic acid.
(13) Signal transduction inhibitors, for example chlozolinate, fenpiclonil, fludioxonil, iprodione, procymidone, quinoxyfen and vinclozolin.
(14) Decouplers, for example binapacryl, dinocap, ferimzone, fluazinam and meptyldinocap.
(15) Further compounds, for example benthiazole, bethoxazin, capsimycin, carvone, chinomethionat, pyriofenone (chlazafenone), cufraneb, cyflufenamid, cymoxanil, cyprosulfamide, dazomet, debacarb, dichlorophen, diclomezine, difenzoquat, difenzoquat methylsulfate, diphenylamine, ecomat, fenpyrazamine, flumetover, fluoromide, flusulfamide, flutianil, fosetyl-aluminum, fosetyl-calcium, fosetyl-sodium, hexachlorobenzene, irumamycin, methasulfocarb, methyl isothiocyanate, metrafenon, mildiomycin, natamycin, nickel dimethyldithiocarbamate, nitrothal-isopropyl, octhilinone, oxamocarb, oxyfenthiin, pentachlorophenol and salts thereof, phenothrin, phosphoric acid and salts thereof, propamocarb-fosetylate, propanosine-sodium, proquinazid, pyrimorph, (2E)-3-(4-tert-butylphenyl)-3-(2-chloropyridin-4-yl)-1-(morpholin-4-yl)prop-2-en-1-one, (2Z)-3-(4-tert-butylphenyl)-3-(2-chloropyridin-4-yl)-1-(morpholin-4-yl)prop-2-en-1-one, pyrrolnitrin, tebufloquin, tecloftalam, tolnifanid, triazoxide, trichlamide, zarilamide, (3S,6S,7R,8R)-8-benzyl-3-[({3-[(isobutyryloxy)methoxy]-4-methoxy-pyridin-2-yl}carbonyl)amino]-6-methyl-4,9-dioxo-1,5-dioxonan-7-yl 2-methylpropanoate, 1-(4-{4-[(5R)-5-(2,6-difluorophenyl)-4,5-dihydro-1,2-oxazol-3-yl]-1,3-thiazol-2-yl}piperidin-1-yl)-2-[5-methyl-3-(trifluoro-methyl)-1H-pyrazol-1-yl]ethanone, 1-(4-{4-[(5S)-5-(2,6-difluorophenyl)-4,5-dihydro-1,2-oxazol-3-yl]-1,3-thiazol-2-yl}piperidin-1-yl)-2-[5-methyl-3-(trifluoro-1-(4-{4-[5-(2,6-difluorophenyl)-4,5-dihydro-1,2-oxazol-3-yl]-1,3-thiazol-2-yl}piperidin-1-yl)-2-[5-methyl-3-(trifluoro-methyl)-1H-pyrazol-1-yl]ethanone, 1-(4-methoxyphenoxy)-3,3-dimethylbutan-2-yl 1H-imidazole-1-carboxylate, 2,3,5,6-tetrachloro-4-(methylsulfonyl)pyridine, 2,3-dibutyl-6-chlorothieno[2,3-d]pyrimidin-4(3H)-one, 2,6-dimethyl-1H,5H-[1,4]dithiino[2,3-c:5,6-cy]dipyrrole-1,3,5,7(2H,6H)-tetrone, 2-[5-methyl-3-(trifluoro-methyl)-1H-pyrazol-1-yl]-1-(4-14-[(5R)-5-phenyl-4,5-dihydro-1,2-oxazol-3-yl]-1,3-thiazol-2-yl]-piperidin-1-yl)ethanone, 2-[5-methyl-3-(trifluoromethyl)-1H-pyrazol-1-yl]-1-(4-{4-[(5S)-5-phenyl-4,5-dihydro-1,2-oxazol-3-yl]-1,3-thiazol-2-yl▾-piperidin-1-yl)ethanone, 2-[5-methyl-3-(trifluoromethyl)-1H-pyrazol-1-yl]-1-14-[4-(5-phenyl-4,5-dihydro-1,2-oxazol-3-yl)-1,3-thiazol-2-yl]-piperidin-1-yl}ethanone, 2-butoxy-6-iodo-3-propyl-4H-chromen-4-one, 2-chloro-5-[2-chloro-1-(2,6-difluoro-4-methoxyphenyl)-4-methyl-1H-imidazol-5-yl]-pyridine, 2-phenylphenol and salts thereof, 3-(4,4,5-trifluoro-3,3-dimethyl-3,4-dihydroisoquinolin-1-yl)-quinoline, 3,4,5-trichloropyridine-2,6-dicarbonitrile, 3-[5-(4-chlorophenyl)-2,3-dimethyl-1,2-oxazolidin-3-yl]pyridine, 3-chloro-5-(4-chlorophenyl)-4-(2,6-difluorophenyl)-6-methylpyridazine, 4-(4-chlorophenyl)-5-(2,6-difluorophenyl)-3,6-dimethylpyridazine, 5-amino-1,3,4-thiadiazole-2-thiol, 5-chloro-N′-phenyl-N′-(prop-2-yn-1-yl)thiophene-2-sulfonohydrazide, 5-fluoro-2-[(4-fluorobenzyl)oxy]pyrimidine-4-amine, 5-fluoro-2-[(4-methylbenzyl) oxy]pyrimidine-4-amine, 5-methyl-6-octyl[1,2,4]triazolo[1,5-a]pyrimidine-7-amine, ethyl (2Z)-3-amino-2-cyano-3-phenylprop-2-enoate, N′-(4-{[3-(4-chlorobenzyl)-1,2,4-thiadiazol-5-yl]oxy}-2,5-dimethylphenyl)-N-ethyl-N-methylimidoformamide, N-(4-chlorobenzyl)-3-[3-methoxy-4-(prop-2-yn-1-yloxy)-phenyl]propanamide, N-[(4-chlorophenyl)(cyano)methyl]-3-[3-methoxy-4-(prop-2-yn-1-yloxy)phenyl]propanamide, N-[(5-bromo-3-chloropyridin-2-yl)methyl]-2,4-dichloropyridine-3-carboxamide, N-[1-(5-bromo-3-chloropyridin-2-yl)ethyl]-2,4-dichloropyridine-3-carboxamide, N-[1-(5-bromo-3-chloropyridin-2-yl)ethyl]-2-fluoro-4-iodopyridine-3-carboxamide, N-{(E)-[(cyclopropylmethoxy)imino][6-(difluoromethoxy)-2,3-difluorophenyl]methyl}-2-phenylacetamide, N-{(Z)-[(cyclopropylmethoxy)imino][6-(difluoromethoxy)-2,3-difluorophenyl]methyl}-2-phenylacetamide, N′-{4-[(3-tert-butyl-4-cyano-1,2-thiazol-5-yl)oxy]-2-chloro-5-methylphenyl}-N-ethyl-N-methylimidoformamide, N-methyl-2-(1-{[5-methyl-3-(trifluoromethyl)-1H-pyrazol-1-yl]-acetyl}piperidin-4-yl)-N-(1,2,3,4-tetrahydronaphthalen-1-yl)-1,3-thiazole-4-carboxamide, N-methyl-2-(1-{[5-methyl-3-(trifluoromethyl)-1H-pyrazol-1-yl]acetyl)-piperidin-4-yl)-N-[(1R)-1,2,3,4-tetrahydronaphthalen-1-yl]-1,3-thiazole-4-carboxamide, N-methyl-2-(1-1[5-methyl-3-(trifluoromethyl)-1H-pyrazol-1-yl]acetyl}-piperidin-4-yl)-N-[(1S)-1,2,3,4-tetrahydronaphthalen-1-yl]-1,3-thiazole-4-carboxamide, pentyl {6-[({[(1-methyl-1H-tetrazol-5-yl) (phenyl)methylidene]amino}oxy)-methyl]pyridin-2-yl}carbamate, phenazine-1-carboxylic acid, quinolin-8-ol, quinolin-8-ol sulfate (2:1) and tert-butyl {6-[({[(1-methyl-1H-tetrazol-5-yl) (phenyl)-methylene]amino}oxy)methyl]pyridin-2-yl}carbamate.
(16) Further compounds, for example 1-methyl-3-(trifluoromethyl)-N-[2′-(trifluoromethyl)biphenyl-2-yl]-1H-pyrazole-4-carboxamide, N-(4′-chlorobiphenyl-2-yl)-3-(difluoromethyl)-1-methyl-1H-pyrazole-4-carboxamide, N-(2′,4′-dichlorobiphenyl-2-yl)-3-(difluoromethyl)-1-methyl-1H-pyrazole-4-carboxamide, 3-(difluoromethyl)-1-methyl-N-[4′-(trifluoromethyl)-biphenyl-2-yl]-1H-pyrazole-4-carboxamide, N-(2′,5′-difluorobiphenyl-2-yl)-1-methyl-3-(trifluoromethyl)-1H-pyrazole-4-carboxamide, 3-(difluoromethyl)-1-methyl-N-[4′-(prop-1-yn-1-yl)biphenyl-2-yl]-1H-pyrazole-4-carboxamide, 5-fluoro-1,3-dimethyl-N-[4′-(prop-1-yn-1-yl)biphenyl-2-yl]-1H-pyrazole-4-carboxamide, 2-chloro-N-[4′-(prop-1-yn-1-yl)biphenyl-2-yl]pyridine-3-carboxamide, 3-(difluoromethyl)-N-[4′-(3,3-dimethylbut-1-yn-1-yl)biphenyl-2-yl]-1-methyl-1H-pyrazole-4-carboxamide, N-[4′-(3,3-dimethylbut-1-yn-1-yl)biphenyl-2-yl]-5-fluoro-1,3-dimethyl-1H-pyrazole-4-carboxamide, 3-(difluoromethyl)-N-(4′-ethynylbiphenyl-2-yl)-1-methyl-1H-pyrazole-4-carboxamide, N-(4′-ethynyl-biphenyl-2-yl)-5-fluoro-1,3-dimethyl-1H-pyrazole-4-carboxamide, 2-chloro-N-(4′-ethynylbiphenyl-2-yl)-pyridine-3-carboxamide, 2-chloro-N-[4′-(3,3-dimethylbut-1-yn-1-yl)biphenyl-2-yl]pyridine-3-carboxamide, 4-(difluoromethyl)-2-methyl-N-[4′-(trifluoromethyl)biphenyl-2-yl]-1,3-thiazole-5-carboxamide, 5-fluoro-N-[4′-(3-hydroxy-3-methylbut-1-yn-1-yl)biphenyl-2-yl]-1,3-dimethyl-1H-pyrazole-4-carboxamide, 2-chloro-N-[4′-(3-hydroxy-3-methylbut-1-yn-1-yl)biphenyl-2-yl]pyridine-3-carboxamide, 3-(difluoromethyl)-N-[4′-(3-methoxy-3-methylbut-1-yn-1-yl)biphenyl-2-yl]-1-methyl-1H-pyrazole-4-carboxamide, 5-fluoro-N-[4′-(3-methoxy-3-methylbut-1-yn-1-yl)-biphenyl-2-yl]-1,3-dimethyl-1H-pyrazole-4-carboxamide, 2-chloro-N-[4′-(3-methoxy-3-methylbut-1-yn-1-yl)-biphenyl-2-yl]pyridine-3-carboxamide, (5-bromo-2-methoxy-4-methylpyridin-3-yl) (2,3,4-trimethoxy-6-methylphenyl)methanone, N-[2-(4-{[3-(4-chlorophenyl)-prop-2-yn-1-yl]oxy}-3-methoxyphenyl)ethyl]-N2-(methyl-sulfonyl)valinamide, 4-oxo-4-[(2-phenylethyl)amino]-butanoic acid and but-3-yn-1-yl {6-[({[(Z)-(1-methyl-1H-tetrazol-5-yl)(phenyl)methylene]amino}oxy)methyl]-pyridin-2-yl}carbamate.

All pesticides (1) to (16) mentioned may, when they are capable on account of their functional groups, optionally form salts with suitable bases or acids.

Examples of bactericides include:
bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulfate and other copper preparations.

Examples of insecticides, acaricides and nematicides include:
(1) Acetylcholinesterase (ACNE) inhibitors, for example
   carbamates, e.g. alanycarb, aldicarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, triazamate, trimethacarb, XMC and xylylcarb; or
   organophosphates, e.g. acephate, azamethiphos, azinphos-ethyl, azinphos-methyl, cadusafos, chlorethoxyfos, chlorfenvinphos, chlormephos, chlorpyrifos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothiophosphoryl) salicylate, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, triclorfon and vamidothion.
(2) GABA-gated chloride channel antagonists, for example
   cyclodiene organochlorines, e.g. chlordane and endosulfan; or
   phenylpyrazoles (fiproles), e.g. ethiprole and fipronil.
(3) Sodium channel modulators/voltage-gated sodium channel blockers, for example
   pyrethroids, e.g. acrinathrin, allethrin, d-cis-trans allethrin, d-trans allethrin, bifenthrin, bioallethrin, bioallethrin s-cyclopentenyl isomer, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin [(1R)-trans isomers], deltamethrin, empenthrin [(EZ)-(1R) isomers], esfenvalerate, etofenprox, fenpropathrin, fenvalerate, flucythrinate, flumethrin, tau-fluvalinate, halfenprox, imiprothrin, kadethrin, permethrin, phenothrin [(1R)-trans isomer], prallethrin, pyrethrins (pyrethrum), resmethrin, silafluofen, tefluthrin, tetramethrin, tetramethrin [(1R) isomers)], tralomethrin and transfluthrin; or DDT; or methoxychlor.
(4) Nicotinergic acetylcholine receptor (nAChR) agonists, for example
   neonicotinoids, e.g. acetamiprid, dinotefuran, imidacloprid, nitenpyram, thiacloprid and thiamethoxam; or
   nicotine.
(5) Allosteric activators of the nicotinergic acetylcholine receptor (nAChR), for example spinosyns, e.g. spinetoram and spinosad.
(6) Chloride channel activators, for example avermectins/milbemycins, e.g. abamectin, emamectin benzoate, lepimectin and milbemectin.
(7) Juvenile hormone imitators, for example juvenile hormone analogs, e.g. hydroprene, kinoprene and methoprene; or
   fenoxycarb; or pyriproxyfen.
(8) Active ingredients with unknown or nonspecific mechanisms of action, for example
   alkyl halides, e.g. methyl bromide and other alkyl halides; or
   chloropicrin; or sulfuryl fluoride; or borax; or tartar emetic.
(9) Selective antifeedants, e.g. pymetrozine; or flonicamid.
(10) Mite growth inhibitors, e.g. clofentezine, hexythiazox and diflovidazin; or etoxazole.
(11) Microbial disruptors of the insect gut membrane, e.g. Bacillus thuringiensis subspecies israelensis, Bacillus sphaericus, Bacillus thuringiensis subspecies aizawai, Bacillus thuringiensis subspecies kurstaki, Bacillus thuringiensis subspecies tenebrionis, and BT plant proteins: Cry1Ab, Cry1Ac, Cry1Fa, Cry2Ab, mCry3A, Cry3Ab, Cry3Bb, Cry34/35Ab1.
(12) Oxidative phosphorylation inhibitors, ATP disruptors, for example diafenthiuron; or
   organotin compounds, e.g. azocyclotin, cyhexatin and fenbutatin oxide; or
   propargite; or tetradifon.
(13) Oxidative phosphorylation decouplers that interrupt the H proton gradient, for example chlorfenapyr, DNOC and sulfluramid.
(14) Nicotinergic acetylcholine receptor antagonists, for example bensultap, cartap hydrochloride, thiocyclam, and thiosultap-sodium.
(15) Chitin biosynthesis inhibitors, type 0, for example bistrifluron, chlorfluazuron, diflubenzuron, flucycloxuron, flufenoxuron, hexaflumuron, lufenuron, novaluron, noviflumuron, teflubenzuron and triflumuron. p1 (16) Chitin biosynthesis inhibitors, type 1, for example buprofezin.
(17) Moulting disruptors, dipteran, for example cyromazine.
(18) Ecdysone receptor agonists, for example chromafenozide, halofenozide, methoxyfenozide and tebufenozide.
(19) Octopaminergic agonists, for example amitraz.
(20) Complex-III electron transport inhibitors, for example hydramethylnon; or acequinocyl; or fluacrypyrim.
(21) Complex-I electron transport inhibitors, for example
   METI acaricides, e.g. fenazaquin, fenpyroximate, pyrimidifen, pyridaben, tebufenpyrad and tolfenpyrad; or
   rotenone (Derris).
(22) Voltage-gated sodium channel blockers, for example indoxacarb; or metaflumizone.
(23) Inhibitors of acetyl-CoA carboxylase, for example tetronic and tetramic acid derivatives, e.g. spirodiclofen and spiromesifen.
(24)—IV electron transport inhibitors, for example phosphines, e.g. aluminum phosphide, calcium phosphide, phosphine and zinc phosphide; or cyanide.
(25) Complex-II electron transport inhibitors, for example cyenopyrafen.
(26) Ryanodine receptor effectors, for example diamides, e.g. chlorantraniliprole and flubendiamide.

Further active ingredients with an unknown mechanism of action, for example amidoflumet, azadirachtin, benclothiaz, benzoximate, bifenazate, bromopropylate, chinomethionat, cryolite, cyantraniliprole (Cyazypyr), cyflumetofen, dicofol, diflovidazin, fluensulfone, flufenerim, flufiprole, fluopyram, fufenozide, imidaclothiz, iprodione, pyridalyl, pyrifluquinazon and iodomethane; and additionally preparations based on Bacillus firmus (I-1582, BioNeem, Votivo) and the following known active compounds:

3-bromo-N-{2-bromo-4-chloro-6-[(1-cyclopropylethyl)-carbamoyl]phenyl}-1-(3-chloropyridin-2-yl)-1H-pyrazole-5-carboxamide (known from WO2005/077934), 4-{[(6-bromopyrid-3-yl)methyl](2-fluoroethyl)amino}furan-2(5H)-one (known from WO2007/115644), 4-{[(6-fluoropyrid-3-yl)methyl](2,2-difluoroethyl)amino}furan-2(5H)-one (known from WO2007/115644), 4-{[(2-chloro-1,3-thiazol-5-yl)methyl](2-fluoroethyl)amino}furan-2(5H)-one (known from WO2007/115644), 4-{[(6-chloropyrid-3-yl)methyl](2-fluoroethyl)amino}furan-2(5H)-one (known from WO2007/115644), 4-{[(6-chloropyrid-3-yl)methyl](2,2-difluoroethyl)amino}furan-2(5H)-one (known from WO2007/115644), 4-{[(6-chloro-5-fluoropyrid-3-yl)methyl](methyl)amino}furan-2(5H)-one (known from WO2007/115643), 4-{[(5,6-dichloropyrid-3-yl)methyl](2-fluoroethyl)amino}furan-2(5H)-one (known from WO2007/115646), 4-{[(6-chloro-5-fluoropyrid-3-yl)methyl](cyclopropyl)amino}furan-2(5H)-one (known from WO2007/115643), 4-{[(6-chloropyrid-3-yl)-methyl](cyclopropyl)amino}furan-2(5H)-one (known from EP-A-0 539 588), 4-{[(6-chloropyrid-3-yl)-methyl]-(methyl)amino}furan-2(5H)-one (known from EP-A-0 539 588), {[1-(6-chloropyridin-3-yl)ethyl](methyl)-oxido-λ⁴-sulfanylidene}cyanamide (known from WO2007/149134) and its diastereomers {[(1R)-1-(6-chloropyridin-3-yl)ethyl](methyl)oxido-λ⁴-sulfanylidene}cyanamide (A) and {[(1S)-1-(6-chloropyridin-3-yl)ethyl](methyl)oxido-λ⁴-sulfanylidene}cyanamide (B) (likewise known from WO2007/149134), and also sulfoxaflor (likewise known from WO2007/149134) and its diastereomers [(R)-methyl(oxido){(1R)-1-[6-(trifluoromethyl)pyridin-3-yl]ethyl }-λ⁴-sulfanylidene]cyanamide (A1) and [(S)-methyl(oxido){(1S)-1-[6-(trifluoromethyl)pyridin-3-yl]ethyl}-λ⁴-sulfanylidene]cyanamide (A2), referred to as diastereomer group A (known from WO 2010/074747, WO 2010/074751), [(R)-methyl(oxido){(1S)-1-[6-(trifluoromethyl)pyridin-3-yl]ethyl}-λ⁴-sulfanylidene]-cyanamide (B1) and [(S)-methyl(oxido){(1R)-1-[6-(trifluoromethyl)pyridin-3-yl]ethyl}-λ⁴-sulfanylidene]-cyanamide (B2), referred to as diastereomer group B (likewise known from WO 2010/074747, WO 2010/074751) and 11-(4-chloro-2,6-dimethylphenyl)-12-hydroxy-1,4-dioxa-9-azadispiro[4.2.4.2]tetradec-11-en-10-one (known from WO2006/089633), 3-(4′-fluoro-2,4-dimethylbiphenyl-3-yl)-4-hydroxy-8-oxa-1-azaspiro[4.5]dec-3-en-2-one (known from WO2008/067911), 1-{2-fluoro-4-methyl-5-[(2,2,2-trifluoroethyl)sulfinyl]phenyl}-3-(trifluoro-methyl)-1H-1,2,4-triazol-5-amine (known from WO2006/043635), [(3S,4aR,12R,12aS,12bS)-3-[(cyclo-propylcarbonyl)oxy]-6,12-dihydroxy-4,12b-dimethyl-11-oxo-9-(pyridin-3-yl)-1,3,4,4a,5,6,6a,12,12a,12b-decahydro-2H,11H-benzo[f]pyrano[4,3-b]chromen-4-yl]-methyl cyclopropanecarboxylate (known from WO2008/066153), 2-cyano-3-(difluoromethoxy)-N,N-dimethylbenzenesulfonamide (known from WO2006/056433), 2-cyano-3-(difluoromethoxy)-N-methylbenzenesulfonamide (known from WO2006/100288), 2-cyano-3-(difluoromethoxy)-N-ethylbenzenesulfonamide (known from WO2005/035486), 4-(difluoromethoxy)-N-ethyl-N-methyl-1,2-benzothiazol-3-amine 1,1-dioxide (known from WO2007/057407), N-[1-(2,3-dimethylphenyl)-2-(3,5-dimethylphenyl)ethyl]-4,5-dihydro-1,3-thiazol-2-amine (known from WO2008/104503), {1′-[(2E)-3-(4-chlorophenyl)prop-2-en-1-yl]-5-fluorospiro[indole-3,4′-piperidin]-1(2H)-yl} (2-chloropyridin-4-yl)methanone (known from WO2003/106457), 3-(2,5-dimethylphenyl)-4-hydroxy-8-methoxy-1,8-diazaspiro[4.5]dec-3-en-2-one (known from WO2009/049851), 3-(2,5-dimethylphenyl)-8-methoxy-2-oxo-1,8-diazaspiro[4.5]dec-3-en-4-yl ethyl carbonate (known from WO2009/049851), 4-(but-2-yn-1-yloxy)-6-(3,5-dimethylpiperidin-1-yl)-5-fluoro-pyrimidine (known from WO2004/099160), (2,2,3,3,4,4,5,5-octafluoropentyl)-(3,3,3-trifluoro-propyl)malononitrile (known from WO2005/063094), (2,2,3,3,4,4,5,5-octafluoropentyl)(3,3,4,4,4-penta-fluorobutyl)malononitrile (known from WO2005/063094), 8-[2-(cyclopropylmethoxy)-4-(trifluoromethyl)phenoxy]-3-[6-(trifluoromethyl)pyridazin-3-yl]-3-azabicyclo-[3.2.1]octane (known from WO2007/040280), 2-ethyl-7-methoxy-3-methyl-6-[(2,2,3,3-tetrafluoro-2,3-dihydro-1,4-benzodioxin-6-yl)oxy]quinolin-4-yl methyl carbonate (known from JP2008/110953), 2-ethyl-7-methoxy-3-methyl-6-[(2,2,3,3-tetrafluoro-2,3-dihydro-1,4-benzodioxin-6-yl)oxy]quinolin-4-yl acetate (known from JP2008/110953), PF1364 (CAS reg. no. 1204776-60-2) (known from JP2010/018586), 5-[5-(3,5-dichlorophenyl)-5-(trifluoromethyl)-4,5-dihydro-1,2-oxazol-3-yl]-2-(1H-1,2,4-triazol-1-yl)benzonitrile (known from WO2007/075459), 5-[5-(2-chloropyridin-4-yl)-5-(trifluoromethyl)-4,5-dihydro-1,2-oxazol-3-yl]-2-(1H-1,2,4-triazol-1-yl)benzonitrile (known from WO2007/075459), 4-[5-(3,5-dichlorophenyl)-5-(trifluoromethyl)-4,5-dihydro-1,2-oxazol-3-yl]-2-methyl-N-{2-oxo-2-[(2,2,2-trifluoroethyl)amino]ethyl}-benzamide (known from WO2005/085216), 4-{[(6-chloropyridin-3-yl)methyl](cyclopropyl)amino}-1,3-oxazol-2(5H)-one, 4-{[(6-chloropyridin-3-yl)methyl]-(2,2-difluoroethyl)amino}-1,3-oxazol-2(5H)-one, 4-{[(6-chloropyridin-3-yl)methyl](ethyl)amino}-1,3-oxazol-2(5H)-one, 4-{[(6-chloropyridin-3-yl)methyl]-(methyl)amino}-1,3-oxazol-2(5H)-one (all known from WO2010/005692), NNI-0711 (known from WO2002/096882), 1-acetyl-N-[4-(1,1,1,3,3,3-hexafluoro-2-methoxypropan-2-yl)-3-isobutylphenyl]-N-isobutyryl-3,5-dimethyl-1H-pyrazole-4-carboxamide (known from WO2002/096882), methyl 2-[2-({[3-bromo-1-(3-chloropyridin-2-yl)-1H-pyrazol-5-yl]carbonyl}amino)-5-chloro-3-methylbenzoyl1-2-methylhydrazinecarboxylate (known from WO2005/085216), methyl 2-[2-({[3-bromo-1-(3-chloropyridin-2-yl)-1H-pyrazol-5-yl]carbonyl}amino)-5-cyano-3-methylbenzoyl]-2-ethylhydrazinecarboxylate (known from WO2005/085216), methyl 2-[2-({[3-bromo-1-(3-chloropyridin-2-yl)-1H-pyrazol-5-yl]carbonyl}amino)-5-cyano-3-methylbenzoyl]-2-methylhydrazinecarboxylate (known from WO2005/085216), methyl 2-[3,5-dibromo-2-({[3-bromo-1-(3-chloropyridin-2-yl)-1H-pyrazol-5-yl]carbonyl}amino)benzoyl]-1,2-diethylhydrazine-carboxylate (known from WO2005/085216), methyl 2-[3,5-dibromo-2-({[3-bromo-1-(3-chloropyridin-2-yl)-1H-pyrazol-5-yl]carbonyl}amino)benzoyl]-2-ethylhydrazine-carboxylate (known from WO2005/085216), (5RS,7RS;5RS,7SR)-1-(6-chloro-3-pyridylmethyl)-1,2,3,5,6,7-hexahydro-7-methyl-8-nitro-5-propoxy-imidazo[1,2-a]pyridine (known from WO2007/101369), 2-{6-[2-(5-fluoropyridin-3-yl)-1,3-thiazol-5-yl]pyridin-2-yl]pyrimidine (known from WO2010/006713), 2-{6-[2-(pyridin-3-yl)-1,3-thiazol-5-yl]pyridin-2-yl}pyrimidine (known from WO2010/006713), 1-(3-chloropyridin-2-yl)-N-[4-cyano-2-methyl-6-(methylcarbamoyl)phenyl]-3-{[5-(trifluoromethyl)-1H-tetrazol-1-yl]methyl}-1H-pyrazole-5-carboxamide (known from WO2010/069502), 1-(3-chloropyridin-2-yl)-N-[4-cyano-2-methyl-6-(methyl-carbamoyl)phenyl]-3-{[5-(trifluoromethyl)-2H-tetrazol-2-yl]methyl}-1H-pyrazole-5-carboxamide (known from WO2010/069502), N-[2-(tert-butylcarbamoyl)-4-cyano-6-methylphenyl]-1-(3-chloropyridin-2-yl)-3-1[5-(trifluoromethyl)-1H-tetrazol-1-yl]methyl}-1H-pyrazole-5-carboxamide (known from WO2010/069502), N-[2-(tert-butylcarbamoyl)-4-cyano-6-methylphenyl]-1-(3-chloro-pyridin-2-yl)-3-{[5-(trifluoromethyl)-2H-tetrazol-2-yl]methyl}-1H-pyrazole-5-carboxamide (known from WO2010/069502) and (1E)-N-[(6-chloropyridin-3-yl)methyl]-N′-cyano-N-(2,2-difluoroethyl)ethanimidamide (known from WO2008/009360).

The active ingredients referred to here by their common names are known and are described, for example, in the Pesticide Manual (16th ed., British Crop Protection Council 2012) or can be searched for on the Internet (e.g. http://www.alanwood.net/pesticides).

The pesticides of component c) may also be a combination of two or more pesticides. Such combinations are of significance especially when the aim is, for example, to broaden the spectrum of action of the pesticide composition or to better suppress resistances to particular pesticides.

In a preferred embodiment of the invention, the amount of the one or more pesticides of component c) in the compositions of the invention is more than 100 g/L, preferably more than 200 g/L and more preferably more than 300 g/L. These figures are based on the total weight of the pesticide composition of the invention and, in the case of pesticides which are used in the form of their water-soluble salts, are based on the amount of free acid, called the acid equivalent (a.e.).

The pH of the pesticide compositions is typically in the range from 3.5 to 8.5, preferably 4.0 to 8.0 and more preferably 4.5 to 6.5 (measured as a 1% by weight aqueous dilution). The pH is determined primarily by the pH values of the solutions of the aqueous pesticides that are in the form of salts of weak acids. By addition of acids or bases, it is possible to adjust the pH to another value different from the original pH of the mixture.

In a further preferred embodiment of the invention, the pesticide compositions of the invention are in the form of concentrate formulations which are diluted prior to use, especially with water (for example “ready-to-use”, “in-can” or “built-in” formulations), and contain the one or more ammonium salts (I) generally in amounts of 5% to 80% by weight, preferably of 10% to 70% by weight and more preferably of 20% to 60% by weight. These figures are based on the overall concentrate formulation.

The pesticide compositions of the invention are preferably deployed to the fields in the form of spray liquors. The spray liquors are produced by diluting concentrate formulations with a defined amount of water.

In a further preferred embodiment of the invention, the pesticide compositions of the invention are in the form of spray liquors and contain 0.001% to 10% by weight, preferably 0.02% to 3% by weight and more preferably 0.025% to 2% by weight of the one or more ammonium salts (I) of component a).

The invention further relates to the use of the pesticide compositions of the invention for control and/or for abatement of weeds, fungal diseases or insect infestation. Preference is given to the use of the compositions of the invention for control and/or for abatement of weeds.

These uses can preferably also take place in what is known as the tankmix method. In this case, the one or more water-soluble pesticides of component g) and components a) to d) and additionally water may thus also take the form of a “tankmix” preparation. In such a preparation, both the one or more water-soluble pesticides and components a) to d), the latter optionally together with further adjuvants, are present separately from one another. The two preparations are mixed with one another prior to deployment, generally shortly beforehand, giving rise to a pesticide composition of the invention.

The invention also provides a method of controlling harmful organisms, wherein the harmful organism or its habitat is brought into contact with an ammonium salt (I) of the invention or a pesticide composition of the invention. The harmful organisms are preferably unwanted plants, in which case the pesticide is correspondingly a herbicide or a mixture of herbicides.

### WORKING EXAMPLES

### Preparation

### Preparation Example 1

37 g of dicamba were suspended in 30 g of deionized water while stirring. Subsequently, 33 g of N-methyl-glucamine were introduced into the active ingredient suspension while stirring. The result was a clear solution.

### Preparation Example 2

37 g of dicamba were suspended in 30 g of deionized water while stirring. Subsequently, 33 g of dimethyl-glucamine were introduced into the active ingredient suspension while stirring. The result was a clear solution.

### Preparation Example 3

21 g of MCPA were suspended in 58 g of deionized water while stirring. Subsequently, 21 g of N-methyl-glucamine were introduced into the active ingredient suspension while stirring. The result was a clear solution.

### Preparation Example 4

20 g of pelargonic acid were suspended in 55 g of deionized water while stirring. Subsequently, 25 g of N-methylglucamine were introduced into the active ingredient suspension while stirring. The result was a clear solution.

### Preparation Example 5

20 g of pelargonic acid were suspended in 10 g of deionized water while stirring. Subsequently, 25 g of dimethylglucamine were introduced into the active ingredient suspension while stirring. The result was a clear solution.

### Preparation Example 6

0.21 g of mesotrione was suspended in 10 g of deionized water while stirring. Subsequently, 0.11 g of N-methylglucamine was introduced into the active ingredient suspension while stirring. The result was a clear solution.

### Preparation Example 7

0.13 g of nicosulfuron was suspended in 10 g of deionized water while stirring. Subsequently, 0.07 g of N-methylglucamine was introduced into the active ingredient suspension while stirring. The result was a clear solution.

### Preparation Example 8

0.11 g of nicosulfuron was suspended in 10 g of deionized water while stirring. Subsequently, 0.065 g of dimethylglucamine was introduced into the active ingredient suspension while stirring. The result was a clear solution.

### Formulations

The composition of the active ingredient formulations from the preparation examples are summarized in table 1:

TABLE 1

| | Example (w %) | | | | | | | |
|---|---|---|---|---|---|---|---|---|
| Composition | 1 | 2 | 3 | 4 | 5 | 6 | 7 | 8 |
| Dicamba | 37 | 37 | 0 | 0 | 0 | 0 | 0 | 0 |
| MCPA | 0 | 0 | 21 | 0 | 0 | 0 | 0 | 0 |
| Pelargonic acid | 0 | 0 | 0 | 20 | 20 | 0 | 0 | 20 |
| Mesotrione | 0 | 0 | 0 | 0 | 0 | 2 | 0 | 0 |
| Nicosulfuron | 0 | 0 | 0 | 0 | 0 | 0 | 1.3 | 1.1 |
| NMG | 33 | 0 | 21 | 25 | 0 | 1 | 0.7 | 0.65 |
| DMG | 0 | 33 | 0 | 0 | 25 | 0 | 0 | 25 |
| Water | 30 | 30 | 58 | 55 | 55 | 97 | 98 | 98.25 |
| Appearance | Clear | Clear | Clear | Clear | Clear | Clear | Clear | Clear |
| | solution | solution | solution | solution | solution | solution | solution | solution |

NMG = N-methylglucamine
DMG = dimethylglucamine

1. An organic ammonium salt of the formula (I) 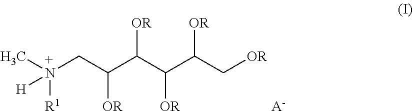
where the symbols have the following definitions:
A⁻ is an anionic pesticide,
R¹ is H, C₁-C₄-alkyl, CH₂CH₂OH or CH₂CH(CH₃)OH;
R is H, CH₃, or two adjacent R radicals together form a —C(R′)— group and
R′ is the same or different and is H or CH₃.

2. An ammonium salt as claimed in claim 1, where A⁻ is the conjugate base of a pesticidal Bronsted acid having a pKₐ in the range from 1.5 to 7, preferably 2 to 6 and more preferably 2.5 to 5.

3. An ammonium salt as claimed in claim 1 or 2, wherein A is selected from the group consisting of: acifluorfen, aminocyclopyrachlor, aminopyralid, amitrol, asulam, benazolin, bentazon, bialafos, bensulfuron, bispyribac, bromacil, bromoxynil, bicyclopyron, chlorflurenol, chlorthal, clodinafop, cloprop, clopyralid, 4-CPA, 4-CPB, 4-CPP, cyhalofop, 2,4-D, dalapon, 2,4-DB, 3,4-DA, 3,4-DB, 3,4-DP, 2,4,5-T, 2,4,5-TB, dicamba, dichlorprop, dichlorprop-P, diflufenzopyr, diclofop, endothal, fenoprop, fenoxaprop, fenthiaprop, flamprop, flamprop-M, florasulam, fluazifop, fluazifop-P, flucarbazone, flufenpyr, flumiclorac, fluoroglycofen, flupropanate, flurenol, fluroxypyr, fluthiacet, fomesafen, foramsulfuron, fosamine, glufosinate, glufosinate-P, glyphosate, halosulfuron, haloxyfop, haloxyfop-P, imizameth, imazamethabenz, imazamox, imazapic, imazapyr, imazaquin, imazethapyr, isoxapyrifop, lactofen, MCPA, MCPA-thioethyl, MCPB, mecoprop, mecoprop-P, mesosulfuron, mesotrione, nicosulfuron, octanoic acid, oleic acid, pelargonic acid, picloram, primisulfuron, propaquizafop, propoxycarbazone, pyraflufen, pyriminobac, pyrithiobac, pyroxsulam, quinclorac, quinmerac, quizalofop, quizalofop-P, sulcotrione, sulfometuron, 2,3,6-TBA, TCA, tembotrione, thiencarbazone, topramezone, tricamba, triclopyr, trifopsic acid, abscisic acid, glyphosine, indole-3-acetic acid, indole-4-butyric acid, jasmonic acid, 1-naphthylacetic acid, 2-naphthylacetic acid, naphthyloxyacetic acid, prohexadione, salicylic acid, 2,3,5-triiodobenzoic acid, trinexapac, thuringiensin, insecticidal free keto-enols such as deacetylated/hydrolyzed spirotetramat or pinoxaden, fosetyl, polyoxorim and polyoxin D.

4. An ammonium salt as claimed in claim 3, wherein A is selected from the group consisting of bicyclopyron, bromoxynil, clopyralid, 2,4-D (2,4-dichlorophenoxyacetic acid), dicamba, diquat, florasulam, fomesafen, glufosinate, glyphosate, MCPA, mesotrione, nicosulfuron, pelargonic acid, quinclorac, quinmerac, salicylic acid, sulcotrione, tembotrione and triclopyr.

5. An ammonium salt as claimed in any of claims 1 to 4, where the symbols in the formula (I) have the following definitions:
R¹ is H, C₁-C₄-alkyl or CH₂CH₂OH and
R is H or CH₃.

6. An ammonium salt as claimed in any of claims 1 to 5, where the symbols in the formula (I) have the following definitions:
R¹ is H, CH₃ or CH₂CH₂OH and
R is H.

7. An ammonium salt as claimed in any of claims 1 to 6, where R¹=CH₃ and R=H or R¹=CH₂CH₂OH and R=H.

8. A process for preparing an ammonium salt as claimed in any of claims 1 to 7, wherein the protonated form of an anionic herbicide is reacted with a glucamine of the formula (II) 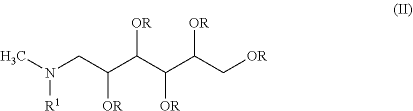
where the symbols have the definitions given in the formula (I).

9. The use of an ammonium salt as claimed in any of claims 1 to 7 as a pesticide.

10. A pesticide composition comprising
a) one or more ammonium salts (I) as claimed in any of claims 1 to 7 and
b) one or more formulation auxiliaries.

11. The pesticide composition as claimed in claim 10 in the form of a herbicide composition.

12. The pesticide composition as claimed in claim 10 or 11, comprising, as formulation auxiliary b), one or more copolymers A) comprise, where the copolymers contain one or more structural units derived from
a) 19.9% to 75.9% by weight of glycerol
b) 0.1% to 30% by weight of at least one dicarboxylic acid and
c) 24% to 80% by weight of at least one monocarboxylic acid of formula (III)
R²—COOH (III)
where R² is (C₅-C₂₉) -alkyl; (C₇-C₂₉)-alkenyl; phenyl or naphthyl.

13. A method of controlling harmful organisms, wherein the harmful organism or its habitat is brought into contact with an ammonium salt (I) as claimed in any of claims 1 to 6 or a pesticide composition as claimed in claim 10 or 11.

14. The method as claimed in claim 13, wherein the harmful organisms are unwanted plants and the pesticide is a herbicide.

15. The use of an ammonium salt as claimed in any of claims 1 to 7 for reducing the liquid in the anionic pesticide present in salt form.
